(19) United States
(12) Patent Application Publication
(10) Pub. No.: US 2014/0213765 A1
(43) Pub. Date: Jul. 31, 2014

(54) Albumin tissue scaffold

(71) Applicant: I-Liang Lee, Zhudong Township (TW)

(72) Inventor: I-Liang Lee, Zhudong Township (TW)

(21) Appl. No.: 13/749,720

(22) Filed: Jan. 25, 2013

(51) Int. Cl. A61L 27/22

(52) U.S. Cl. CPC A61L 27/227
USPC 530/362

(57) ABSTRACT

A tissue scaffold that made of albumin having continuous solid network and void are disclosed. Methods for preparing albumin tissue scaffolds from animal albumins are also disclosed.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISC APPENDIX

Not applicable

### BACKGROUND OF THE INVENTION

Tissue scaffolds are three dimensional porous materials, support cell attachment, growth, and differentiation, directing new tissue formation in vitro or in vivo. Tissue scaffold are useful in tissue engineering developed for replacing damaged human tissues. Many synthetic and native materials have been fabricated into tissue scaffolds, for example plastic polymers, copolymers, metals, proteins, and polysaccharides. Many physical and chemical methods have been applied to generate tissue scaffolds, for examples self-assembly materials, electrospinning, freeze-dry, gas-forming, and emulsification.

Ideal tissue scaffold must have sufficient mechanical strength to maintain its pore structure. The material of ideal tissue scaffold must have cell-adherent property provided binding sites to interact with cells. The void of ideal tissue scaffold allows fluid free diffusion throughout material, which delivers nutrients, growth factors, and cells to every pore. The preferred tissue scaffold should be biodegradable, and to be replaced by new forming tissue. The material and its degraded products have no adverse effects to cells such as necrosis, apoptosis, cell transformation, and carcinogenesis. The material and its degraded products have immunological compatibility by means of no local immune responses and systemic inflammatory responses, and no foreign material responses. The degraded products can be removed via circulation or utilized by cells. The degraded products of preferred materials also have additional advantages uptake by cells as the energy source or as the nutrients. The preferred materials have large pore size that provided sufficient space for cell colony formation, which may facilitate to new tissue formation. The decomposed rate of preferred tissue scaffold should be appropriate, roughly match to the rate of new tissue formation.

It is necessary developed many different kinds of tissue scaffold having distinct mechanical and biological properties giving unique merits that can fulfill various applications and needs in tissue engineering.

Albumin is a plasma protein. Albumin binds fatty acids, steroids, ions, metabolites, hormones, and drugs, served as a molecular carrier to deliver their cargos distributing to whole living body via the circulation. Albumin is also important in maintaining the osmotic pressure of the blood. Most animals have this protein to keep normal physiological function of the circulation.

Kowanko (U.S. Pat. No. 5,385,606) described a method to generate a tissue adhesive in which a di- or polyaldehyde solution uses to cross link an animal derived protein solution formed the adhesive.

Nonaka et al. (Agricultural and Biological Chemistry 53: 2619, 1989) used microbial transglutaminase, a transglutaminase (EC 2.3.2.13) isolated from microbial Streptoverticillium, polymerized human serum albumin and bovine serum albumin solution under a calcium-free buffered solution.

### SUMMARY OF THE INVENTION

The present invention features a tissue scaffold in that the material of animal albumin made of the matter. The present invention also provides methods to generate albumin tissue scaffold from animal albumins included human, bovine, and porcine albumins. Albumin tissue scaffold is a three dimensional porous material with various shapes such as cylinder, cube, and rectangular block having different sizes. The solid of the albumin tissue scaffold comprises a network, and the constitution of network consists of an albumin polymer. The unfilled volume in albumin tissue scaffold comprises a void, gas and liquid can fill up this space. Albumin tissue scaffolds are useful in tissue engineering to provide a framework for cell attachment, proliferation, and new tissue formation.

According to an aspect of the present invention, the albumin tissue scaffold comprises of albumin polymers. Two approaches for synthesizing albumin polymers are demonstrated in this invention, they are chemical agent and cross-linking enzyme. In a chemical polymerization, a chemical polymerizes albumins into albumins. In an enzymatic polymerization, an enzyme polymerizes albumins into albumin polymers. Two classes of albumin polymers are chemically cross-linked albumins and enzymatically cross-linked albumins, both can be applied. Albumin tissue scaffolds have been successful generated from chemically cross-linked albumins and enzymatically cross-linked albumins by using this invention. Other chemicals and enzymes of protein cross linkers have not been demonstrated, and they are not intended to be interpreted as limiting the invention.

In a preferred embodiment, a di-aldehyde cross linker, glutaraldehyde was used. Glutaraldehyde added to a 20% albumin solution at a weight ratio of one part by weight to every 15 to 30 parts by weight of albumin. The albumin polymers obtained by glutaraldehyde cross linking method that is belonging to chemically cross-linked albumins. The related art in this reaction is U.S. Pat. No. 5,385,606.

In a preferred embodiment, a cross-linking enzyme, microbial transglutaminase from microbe Streptoverticillium was used. Microbial transglutaminase added to a 5% albumin solution at the weight ratio of one part by weight to every 100 parts by weight of albumin. The albumin polymers obtained by microbial transglutaminase cross linking method that is belonging to enzymatically cross-linked albumins. The related art in this reaction is Agricultural and Biological Chemistry 53: 2619, 1989.

According to an aspect of the present invention, the resulted polymeric albumin in polymerization reaction is heterogeneous. The presents of albumin oligomers, low molecular weight albumin polymers, and high molecular weight albumin polymers were found in polymeric albumin. High molecular weight albumin polymers, which insoluble in aqueous solution, readily isolated from low molecular weight albumin polymers and albumin oligomers by centrifugation. After polymerization, polymeric albumin was homogenized in a solution by using a homogenizer, and then a centrifugation force of 2,330 g for 5 min was applied to recover high molecular weight albumin polymers. The term “albumin polymers” as used herein when refers to a purified polymerized albumins from a polymerization reaction which comprises essentially high-molecular weight species of polymerized albumin without substantial amounts of un-polymerized and low-molecular weight species.

According to an aspect of the present invention, the porous structure of albumin tissue scaffold is forming during freeze-drying processing. The albumin polymer is transferred into a casting mold, frozen, and then vacuum dried. Tissue culture plates or tissue culture dishes with various shapes and sizes are use as casting molds, most preferably, a 96-well tissue culture plate is used in this invention. The resulted albumin tissue scaffold further treats with a gaseous phase cross linker, formaldehyde. The formaldehyde treatment gives cross links among albumin polymers, fix the shape of albumin tissue scaffold permanently. The vapor of formaldehyde came from a 4% formaldehyde solution and the duration for treatment was about 1 hour.

In a preferred embodiment, the surface of albumin tissue scaffolds showed a porous structure under surface electron microscopic examination. Surface pore size of albumin tissue scaffold is inversely proportional to the degree of albumin cross links. The results of pore geometry measurements have a range of about few μm to about few hundred μm in diameter, more preferably among 42 to 225 μm. These surface pores are large, it would be sufficient for animal cells typically of 10 to 50 μm in diameter to move to these pores without obstruction.

In a preferred embodiment, the inner of the albumin tissue scaffold showed porous structure under surface electron microscopic examination Inner pore size of albumin tissue scaffold is inversely proportional to the degree of albumin cross links. The results of pore geometry measurements have a range of about few μm to about few hundred μm in diameter, the same as to respective surface pore geometry measurements. These inner pores are large, it wound be sufficient for animal cells typically of 10 to 50 μm in diameter to migrate in these pores.

According to some embodiments, the invention features the solid matter of albumin tissue scaffolds having a continuously solid network. The same pore structures from the surface and the inner of albumin tissue scaffold were found. Interstitial connections among pores were also found under surface electron microscopic examination.

In a preferred embodiment, albumin tissue scaffold binds substantial amount of liquid such as water, phosphate-buffered saline, isotonic solutions, and tissue culture mediums. The water bindings of the albumin tissue scaffold have a ratio of from about 16 to about 44, the weight of water divided by the weight of albumin tissue scaffold, which is inversely proportional to the degree of albumin cross link.

In a preferred embodiment, the wet albumin tissue scaffold has resilient property. Contained liquid flows out from albumin tissue scaffold when applied a compressive force to the wet albumin tissue scaffold. The albumin tissue scaffold possesses the ability to recover from a compressive deformation when re-absorbed liquid surround. Under dry condition, the albumin tissue scaffold has shown no significant resilient property. A compressive cyclic testing by mechanical testing machine demonstrated that the albumin tissue scaffold has a full elastic, sponge-like property, to completely recover from a 0.8 compressive strain in a water tank.

In a preferred embodiment, the albumin tissue scaffold supported animal cell attachment. Human mesenchymal stem cells were subcultured to an albumin tissue scaffold. One day after subculturing, bound cells were fixed by 4% paraformaldehyde, dehydrated by acetone, and then revealed by surface electron microscopic examination. A wide range of adherent cells of mammalian origins can be seeded to albumin tissue scaffold. The source of cell is not a limited factor, and may depend on the intent use. A preferred source of cells is select from the group consisting of blood-derived, cord blood-derived, amniotic fluid-derived, skin-derived, adipose-derived, bone marrow-derived, and surgical biopsy-derived somatic cells and stem cells.

The principle constitution of the albumin tissue scaffold is polypeptide, which is degradable via proteolysis to peptide fragments or amino acids, subsequently uptake and utilize by living cells. The invention provides the ways to fabricate this novel tissue scaffold. An albumin having similar amino acid composition, peptide sequence, and tertiary structure from native and recombinant sources is adapted to use the present method.

### DESCRIPTION OF THE DRAWINGS

FIG. 1 is a SEM image of an albumin tissue scaffold prepared by chemically cross-linking albumins with 1:15 weight ratio of glutaraldehyde to albumin.

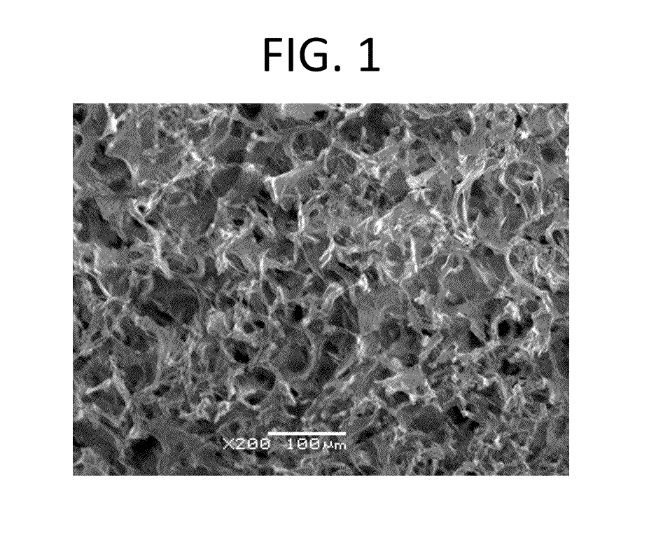

FIG. 2 is a SEM image of an albumin tissue scaffold prepared by chemically cross-linking albumins with 1:20 weight ratio of glutaraldehyde to albumin ratio.

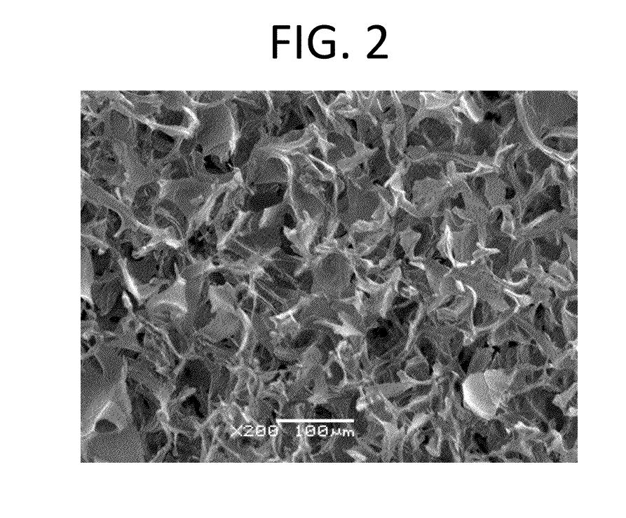

FIG. 3 is a SEM image of an albumin tissue scaffold prepared by chemically cross-linking albumins with 1:25 weight ratio of glutaraldehyde to albumin.

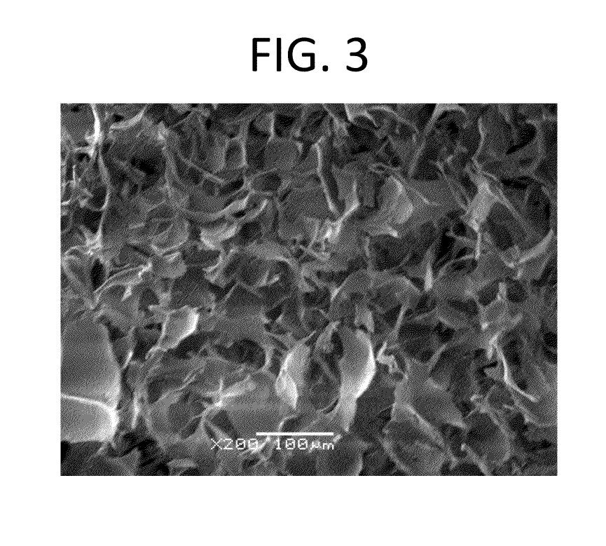

FIG. 4 is a SEM image of an albumin tissue scaffold prepared by chemically cross-linking albumins with 1:30 weight ratio of glutaraldehyde to albumin ratio.

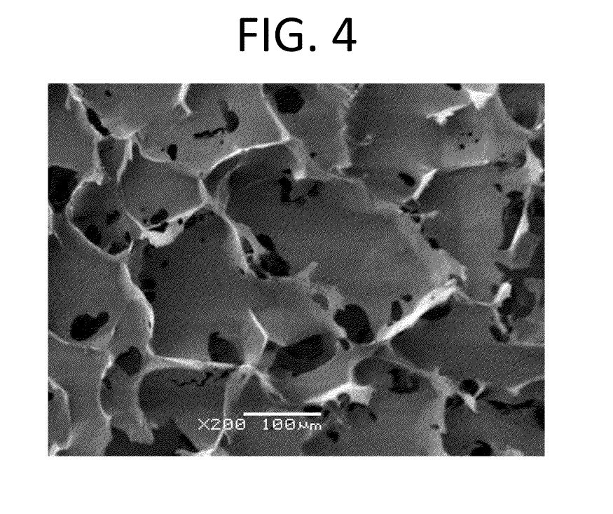

FIG. 5 is a SEM image of an albumin tissue scaffold prepared by enzymatically cross-linking albumins with 1:100 weight ration of microbial transglutaminase to albumin.

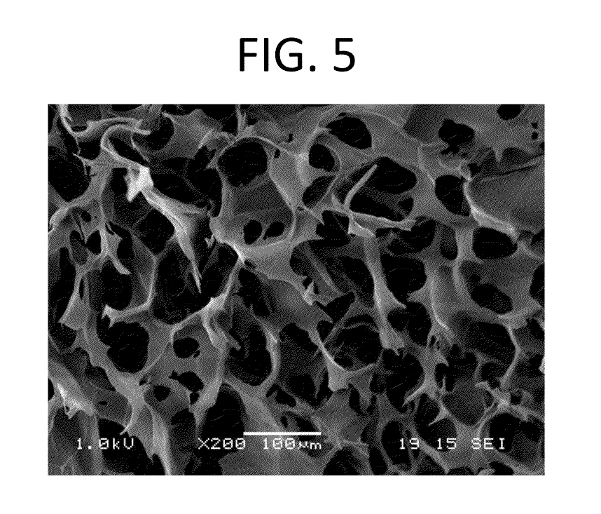

FIG. 6 is the inner structure of the FIG. 5 sample.

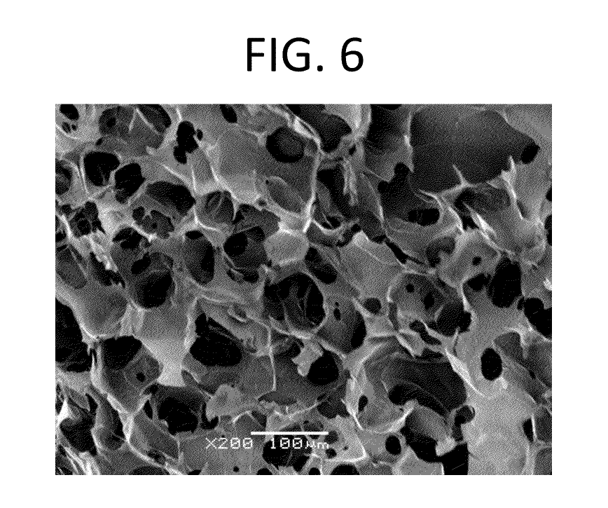

FIG. 7 is the result of a cyclic compressive test for an albumin tissue scaffold in water tank.

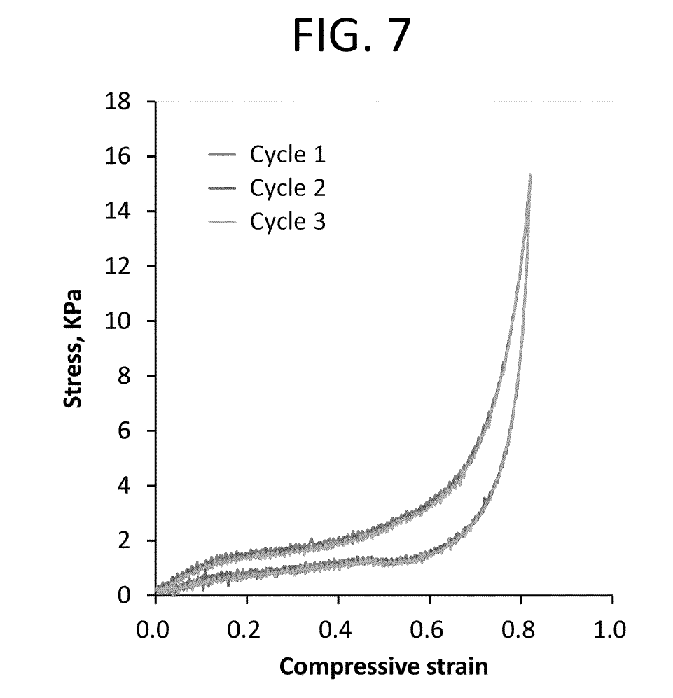

FIG. 8 is a SEM image of a MSC-seeded albumin tissue scaffold.

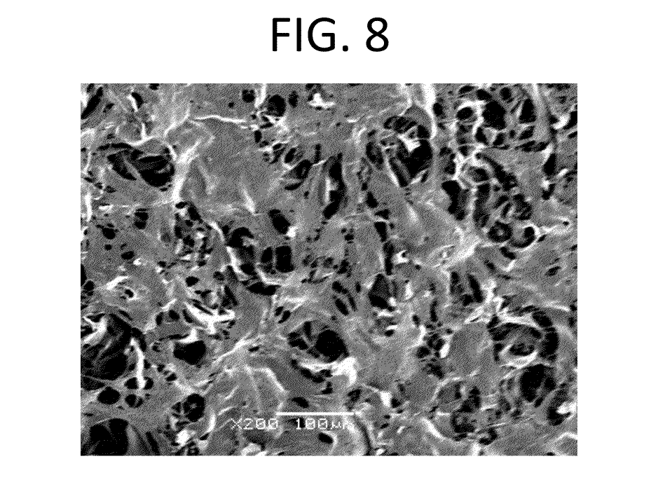

### DESCRIPTION OF PREFERRED EMBODIMENTS

The tissue scaffold having a continuous solid network. The solid network of tissue scaffold consists of a polymer of albumin protein prepared from polymerization reaction. There are two preparative methods, chemical crosslinker-catalyzed polymerization reaction and transglutaminase-catalyzed polymerization reaction, both can generate polymeric albumin. The preferred animal albumin is selected from the group consisting of bovine albumin, human albumin, and porcine albumin. The polymerization reactions preferably have mild conditions in which no organic solvents, 100% aqueous phase, neutral pH value, mild buffer and salt strengths, no excess heat generation during polymerization reaction, no heating requirement, and no chaotropic agent.

Commercial available albumins from animals are provided in dried and lyophilized powders. These powders were dissolved in a suitable reaction buffer to make an albumin solution. The preferred buffer substance is selected from the group consisting of BICINE, HEPES, MOPS, and TRIS. In a chemically cross linking reaction, a di-aldehyde was added to the albumin solution. In an enzymatically cross linking reaction, a transglutaminase was added to the albumin solution.

The polymerization reaction was carried out at the temperature of 37° C. Extensive cross links among individual albumin molecules occurred during incubation. The proceeding of polymerization can be traced using stirring. The reaction, at first, became high viscous, and then it turned into a solid form. The time required for curing solution is vary, which greatly depend on the amounts of cross linkers and albumin that are used. The preferred time for reaction incubation is between 0.5 to 24 hours.

In the present invention, it was found that not all albumins will be incorporated into high molecular weight polymers after polymerization reaction. Some albumins have shown to un-polymerization or low degree of polymerization. The components of polymeric albumin is typically assay by using SDS-PAGE analysis. A denaturing solution and a mechanical homogenizer are applied for disrupting noncovalent protein-protein interactions among albumin polymers. The preferred denatured agents are urea and guanidine. The preferred mechanical homogenization method is selected from the group consisting of pipetting, chopping and mincing, French press, pestle homogenizer, motor-driven tissue homogenizer, and warning blender.

In the present invention, it has found that centrifugation can effectively recover high molecular weight albumin polymers from the polymerization reaction. High molecular weight albumin polymers are insoluble, can be pelleted by centrifugation at about 2,330 g force for about 5 min. Albumin oligomers and low molecular weight albumin polymers remain in the supernatant.

In the present invention, albumin polymers comprise high molecular weight albumin polymers which is essential free of low molecular weight albumin polymer and albumin oligomers. The albumin polymer can be prepared from an enzymatic or a chemical polymerization reaction.

In the present invention, the albumin polymer is subject to wash by a diluted solution before freeze-drying. The preferred substance is pure water or a diluted acid solution which selected from the group consisting of formic acid, acetic acid, lactic acid and citric acid. The washed albumin polymer was transferred into a casting mold, frozen in low temperature, and then freeze-drying. A freeze-dryer can maintain the vacuum under less than 100 mtorr of pressure is used.

In the present invention, vaporous formaldehyde was used to cross link among the albumin polymers. Formaldehyde treatment fixes the shape and the size of albumin tissue scaffold.

### Example 1

2 g bovine serum albumin (purity>98%; Sigma) was dissolved in 19 mL buffer of 50 mM BICINE, pH 8.3. The solution was concentrated by using a spin concentrator (GE Healthcare) to the final volume of 10 mL. Albumin solution was stored in 4° C. refrigerator. Diluted glutaraldehyde regents at the concentrations of 25%, 12.5%, 6.25%, 3.13%, 1.56, and 0.78% were fresh made from 50% glutaraldehyde solution (Sigma) and pure water (Millipore). The reagents were kept on ice to prevent the spontaneous degradation of very diluted glutaraldehyde solution. 0.020 mL of various concentrations of glutaraldehyde was combined with 0.180 mL of bovine serum albumin solution in fresh plastic tube, mixed up immediately by a vortex mixer at top speed. Samples were incubated at 37° C. The following observations were noted after 30 min incubation:

TABLE 1

| Weight % glutaraldehyde | Observation |
|---|---|
| 50 | solid state |
| 25 | solid state |
| 12.5 | solid state |
| 6.25 | solid state |
| 3.13 | liquid state |
| 1.56 | liquid state |
| 0.78 | liquid state |
| 0 | liquid state |

### Example 2

Samples in EXAMPLE 1 were return to the incubator, and an additional incubation of 11.5 hours was performed. The state of each sample was the same as before. 3.4 mL of 8 M urea solution were added to every sample. For those solid state samples, the content was transferred to a tissue grinder (Kontes), and then homogenized by a homogenizer (IKA) at the rotational speed of 2000 rpm for several strokes. The homogenization was keep on ice during processing to prevent sample overheat. For those liquid state samples, content was mixed by a vortex mixer. The resulted homogenates were analyzed by SDS-PAGE analysis. NuPAGE LDS sample buffer (Life Technologies) included reducing agent was added, and then loaded to NuPAGE Bis-Tris Mini gel (Life Technologies). After electrophoresis, gel was stained with Instant blue (Novexin) to reveal protein bands. Following observation were noted after gel stain:

TABLE 2

| Weight % glutaraldehyde | Observation |
|---|---|
| 50 | High molecular weight polymer |
| 25 | High molecular weight polymer |
| 12.5 | High molecular weight polymer |
| 6.25 | High molecular weight polymer, low molecular |
| | weight polymer, and oligomers |
| 3.13 | High molecular weight polymer, low molecular |
| | weight polymer, and oligomers |
| 1.56 | High molecular weight polymer, low molecular |
| | weight polymer, and oligomers |
| 0.78 | Low molecular weight polymer, and oligomers |
| 0 | Oligomers |

### Example 3

Preparation of the albumin tissue scaffold was done as follows. 2 g bovine serum albumin, purity>98% purchased from Sigma, was dissolved in 8.8 mL buffer of 50 mM BICINE, pH 8.3. The albumin solution was kept in 4° C. refrigerator. 0.026, 0.020, 0.016, and 0.013 mL of 50% glutaraldehyde solution were combined with 1 mL of albumin solution in tubes which correspond to 1:15, 1:20, 1:25, and 1:30 weight ratio of glutaraldehyde to albumin, respectively. Samples were incubated at 37° C. for 2 hours. 40 mL of the ice-cold solution of 6 M urea, 0.1 M sodium acetate, pH 5.0 was added to each sample, and then homogenized. The resulted homogenate was centrifuged at 2,330 g for 5 min. The pellets, which containing high molecular weight albumin polymers, were recovered in every sample. 40 mL of 0.1% lactic acid (Sigma) was added to suspend the albumin polymers, incubated on room temperature for 5 min, and then pelleted by centrifugation 2,330 g for 5 min. The lactic acid washing step was repeated more twice to remove urea from albumin polymers. A volume of 0.1 mL of albumin polymer was transferred to 96-well culture plate (Falcon) using a positive-displacement pipette (Gilson). The plate was kept in a −80° C. deep freezer (Thermo) for 1 hour, then moved to a freeze dryer (VirTis) for 24 hours. The porous scaffold was obtained after freeze-drying. The plate was placed in a 2.5-L container included 250 mL of 4% paraformaldehyde (Sigma) in the bottom of container. The vaporous cross linking treatment was performed at room temperature for 1 hour. Prepared tissue scaffold was then stored in a dry box.

### Example 4

Scanning electron microscopes. Albumin tissue scaffolds were mounted onto sample holder using a conductive tape (EMS). Samples were coated by gold and observed under SEM (JEOL). For observing inner structure, used albumin tissue scaffolds were saved after surface examination, horizontally cut through the center by a blade (Leica) into the half. Surface pore diameters were estimated as followings:

TABLE 3

| Weight ratio | Pore size in diameter, μm |
|---|---|
| 15 | 57 ± 15 |
| 20 | 76 ± 17 |
| 25 | 99 ± 19 |
| 30 | 174 ± 51 |

### Example 5

Water binding. Albumin tissue scaffold was soaked in pure water (Millipore), and then determined the wet weight. A filter paper (Whatman) was used to blot off the water from wet albumin tissue scaffold to semi-dry, and then placed the samples in a 60° C. oven for 2 hours. The dried weight of dehydrated sample was then determined. The water binding was calculated as the weight ratio that divided the wet weight by the dried weight. The following results were obtained:

TABLE 4

| Weight ratio | Water binding |
|---|---|
| 15 | 17 ± 1.5 |
| 20 | 26 ± 0.7 |
| 25 | 38 ± 2.5 |
| 30 | 42 ± 2.1 |

### Example 6

Cyclic compressive test. Sample was rinse by Milli Q water. Sample was placed in a 3-cm tissue culture dish contained 1 mL of the Milli Q water. A cyclic compressive testing was setup and performed at ambient by a testing machine (Instron).

### Example 7

Cell adhesion. Albumin tissue scaffold was soaked in pure water (Millipore), washed by Dulbecco's PBS (Invitrogen) three changes, and then culture medium three changes (Invitrogen). A cell suspension of MSC (Cambrex) was prepared in the culture medium at the density of 1e6 cells per mL. 10 μL of cell suspension was transferred onto the prepared albumin tissue scaffold. After 24 hour incubation, sample was washed by Dulbecco's PBS three times, and then fixed by 4% paraformaldehyde/PBS for 1 hour at room temperature. Sample was soak in 6.8% sucrose/PBS overnight, dehydrated by acetone, and the dried by critical point dryer (Tousimis). Samples were coated by gold and observed under SEM (JEOL).

### Example 8

Preparation of the albumin polymer was done as follows. 0.05 g human, bovine, or porcine serum albumin (purity>98%, all from Sigma) was dissolved in 0.475 mL of 50 mL BICINE, pH 8.3 buffer. Polymerization reaction was carried out by adding 0.5 mL of 1 mg/mL microbial transglutaminase (AJINOMATO) and 0.025 mL of 0.5 M DTT (Sigma) into albumin solution. The reaction was incubated at 37° C. for 18 hr. The resulted albumin solid was homogenized in 9 mL of 6 M urea, 0.1 M sodium acetate, pH 5.0. The homogenate was spin down at 2,330 g for 5 min, and the supernatant was discarded. 9 mL of 0.1% lactic acid was added to suspend the pelleted albumin polymers. The suspension was spin down at 2330 g for 5 min. The lactic acid washing step was repeated more twice. A volume of 0.1 mL of albumin polymer was transferred to 96-well culture dish. The plate was frozen at −80° C. for 1 hour, subsequently moved to freeze dryer for 24 hours. After freeze-drying, porous tissue scaffolds was generated. The plate was then placed in a 2.5-L sealed container included 250 mL of 4% paraformaldehyde. The cross linking treatment was performed at room temperature about 25° C. for 1 hour. Prepared tissue scaffold was then stored in dry box. The examinations revealed that the sponge have following characterizations: pore diameter between about 54 μm to about 124 μm, water binding of about 43.4±1.5, and having resilient property in water.

What is claimed is:

1. A tissue scaffold having substantially continuous solid network and voids comprised of an albumin polymer.

2. The tissue scaffold of claim 1, wherein said albumin polymer comprised of a chemically cross linked albumins.

3. The tissue scaffold of claim 1, wherein said albumin polymer comprised of an enzymatically cross linked albumins.

4. The tissue scaffold of claim 1, wherein said albumin is human albumin.

5. The tissue scaffold of claim 1, wherein said albumin is bovine albumin.

6. The tissue scaffold of claim 1, wherein said albumin is porcine albumin.

7. The tissue scaffold of claim 1, wherein said albumin is animal albumins.

8. The tissue scaffold of claim 1, wherein said albumin is recombinant albumins.

9. The tissue scaffold of claim 1, wherein said tissue scaffold is three dimensional.

10. The tissue scaffold of claim 1, wherein said tissue scaffold is porous.

11. The tissue scaffold of claim 1, wherein said tissue scaffold is liquid absorption.

12. The tissue scaffold of claim 1, wherein said tissue scaffold is resilient in liquid solution.

13. The tissue scaffold of claims 11 and 12, wherein said liquid is water, physiological buffered saline, isotonic solutions, and culture mediums.

14. The tissue scaffold of claim 1, wherein said void is cell permeable.

15. The tissue scaffold of claim 1, wherein said solid network is cell adherent.

16. The tissue scaffold of claims 14 and 15, wherein said animal cells are somatic cells derived from animal blood and tissues.

17. The tissue scaffold of claims 14 and 15, wherein said animal cells are stem cells derived from animal blood and tissues.

18. The tissue scaffold of claim 1, wherein said tissue scaffold is degradable.
